(19) United States
(12) Patent Application Publication
(10) Pub. No.: US 2010/0168799 A1
(43) Pub. Date: Jul. 1, 2010

(54) ULNAR OSTEOTOMY PLATE INCLUDING INCREASED COMPRESSION

(76) Inventor: Evan D. Schumer, Newton, MA (US)

Correspondence Address: MARK TERRY, ESQ., 801 BRICKELL AVE., SUITE 900, Miami, FL, 33131, US

(21) Appl. No.: 12/345,405

(22) Filed: Dec. 29, 2008

(51) Int. Cl. A61B 17/70; A61B 17/04

(52) U.S. Cl. 606/286; 606/280; 606/301

(57) ABSTRACT

The present invention provides an ulnar osteotomy plate for use in an ulnar shortening osteotomy. The ulnar osteotomy plate includes an elongated metallic plate having tapered ends, wherein the plate is concave in a direction perpendicular to a main axis. The ulnar osteotomy plate further includes a plurality of holes along the main axis, wherein the plurality of holes includes a substantially elliptical hole with a beveled interior edge located on a first side of the plate and a threaded hole located on a second side of the plate. A first screw is inserted into one end of the elliptical hole and further into an ulna underneath the plate, wherein the first screw includes a beveled edge underneath a head. A second screw is inserted into the threaded hole and further into the ulna, wherein the second screw includes a portion of the shaft near a head to be threaded.

### FIELD OF THE INVENTION

The invention disclosed broadly relates to the field of medical implants, and more particularly relates to medical implants used for ulnar osteotomies.

### BACKGROUND OF THE INVENTION

An osteotomy, well known in the orthopaedic arts, is a surgical operation whereby a bone is cut to shorten, lengthen, or change its alignment. An osteotomy consists of the cutting of a portion of bone and the joining of the resulting pair of free ends of bone. First, the bone is cut in a precise way to correct whatever deformity or congenital problem exists. Secondly, the two newly cut ends of the bone must be rigidly affixed to allow a solid bone mass to form between them during the healing process.

Typically, bone cuts in an osteotomy are accomplished either free hand, or with the use of a “jig” or other cutting guide that is applied to the bone and then removed after the cuts have been created. Once this has been accomplished, the cut bone ends are rigidly held together, ideally in compression, which necessitates the application of a second device such as an implantable plate that would keep the bone ends affixed during the healing process. One such type of osteotomy is an ulnar shortening osteotomy.

An ulnar shortening osteotomy is a well-recognized procedure for the treatment of pain caused by ulnar impaction, which may occur as an isolated entity or in combination with other skeletal derangements of the wrist joint such as fracture of the radius, disruption of the distal radioulnar joint, triangular fibrocartilage tears, and so forth. The method of performing an ulnar shortening osteotomy consists of cutting a portion of the ulna bone for removal (either freehand or using a cutting guide or jig), followed by immobilization of the ulna with a compression plate attached to the bone with screws. Various problems, however, can arise during an ulnar shortening osteotomy.

One problem associated with performing an ulnar shortening osteotomy freehand (without the use of a cutting guide) involves maintaining the bone in a fixed position during cutting such that the two remaining surfaces of the cut bone are uniform and planar. When no cutting guide is used, it can be difficult to hold the cutting tool and the bone in place using just the surgeon's hands. When the surgeon proceeds to reattach the two ends of bone freehand, the two ends must be aligned and kept from rotating, so as to ensure a proper attachment position. Again, this can be challenging using only a surgeon's hands.

Keeping the cutting tool and bone stationary, as well as alignment of the ends of cut bone, can also be a problem when a cutting guide is used. Another problem associated with using a cutting tool is the increased number of devices or parts necessary to perform the ulnar shortening osteotomy. Typically, the cutting guide is attached to a plate or other device, which is secured to the bone. Then, the cutting guide is removed and a separate plate is attached to the cut bone ends so as to couple and compress them. The increased number of parts used in this process can be complicated for the surgeon and increases the risk of misuse and/or user error.

With regard to the attachment of the cutting guide to a plate or other device, which is secured to the bone, it is necessary to maintain an extremely secure attachment because of the vibrations caused by the cutting tool when it comes into contact with the cutting guide. One approach to this problem includes the use of cylindrical protrusions on the cutting guide, which are inserted in similarly shaped bores in the plate or other device, which is secured to the bone. Although this approach provides a level of attachment, the cutting guide may still fall out when vibrations are experienced. Thus, a more secure or tight connection is sought between the cutting guide and the plate or other device, which is secured to the bone.

When reattaching the two cut bone ends using a plate, compression of the two ends of bone can be a problem. In order to ensure proper healing of two cut bone ends, it is necessary to provide a high level of compression between the two bone ends so as to foster the formation of bone mass at the junction point. Screws that attach to the plate to the bone are typically positioned perpendicularly or near-perpendicularly to the bone. This arrangement may provide compression between the plate and the bone, but does not provide much compression along the axis of the bone. Further, this arrangement will attach the plate to the bone, but will not necessarily provide any compression along the axis of the bone.

When using a cutting jig and a plate, it is often necessary to drill holes in the ulna which are not filled at the end of the ulnar shortening osteotomy. For example, when the plate is initially attached to the bone before the cutting occurs, a first screw is often screwed into a first end of the bone so as to secure the plate to the bone. After the cutting occurs, the first screw is removed so that the first end of bone can be moved towards the second end of bone and compressed together. Often, the first screw is either not reused or placed in a new location. This leaves a hole in the bone where the first screw was originally attached. This is disadvantageous since a hole in the ulna can weaken the bone, lead to fracture or cause other complications.

Further, when using a cutting jig and a plate, it is often difficult or impossible to reach the section of bone underneath the plate when cutting the ulna. Because a plate is closely fitted to follow the contour of the ulna, and a cutting jig placed to the side and lower than the plate, it can be challenging to reach the portion of bone just beneath the plate. This can cause problems and delays during healing of the ulnar osteotomy as different plate designs have been advocated so as to reach this section of bone so as to cut it.

Another problem with ulnar osteotomies involves the placement of the plate. Often, the plate is attached to the ulna on the outside side of the bone. This location of the human arm is subject to much contact and impact during everyday use. Thus, patients with a plate in this area may complain of irritation, bruising and pain, thereby necessitating removal of the plate when the bone is completely healed. The need for a second surgery to remove the plate is disadvantageous due to the risks associated with any surgery.

Therefore, a need exists to overcome the problems with the prior art as discussed above, and particularly for a more efficient way to perform an ulnar shortening osteotomy and reduce negative side effects of the surgery.

### SUMMARY OF THE INVENTION

Briefly, according to an embodiment of the present invention, an ulnar osteotomy plate for use in an ulnar shortening osteotomy is disclosed. The ulnar osteotomy plate includes an elongated metallic plate having tapered ends, wherein the plate is concave in a direction perpendicular to a main axis of the plate. The ulnar osteotomy plate further includes a plurality of holes along the main axis of the plate, wherein the plurality of holes includes a substantially elliptical hole with a beveled interior edge located on a first side of the plate and a threaded hole located on a second side of the plate.

In another embodiment of the present invention, a system for performing an ulnar shortening osteotomy is disclosed. The system includes an elongated metallic plate having tapered ends, wherein the plate is concave in a direction perpendicular to a main axis of the plate. The ulnar osteotomy plate includes a plurality of holes along the main axis of the plate, wherein the plurality of holes includes a substantially elliptical hole with a beveled interior edge located on a first side of the plate and a threaded hole located on a second side of the plate. The system also includes a first screw for insertion into one end of the elliptical hole and further into an ulna underneath the plate, wherein the first screw includes a beveled edge underneath a head such that when the beveled edge of the first screw contacts the beveled interior edge of the elliptical hole, there results a force along the main axis towards the first side. The system also includes a second screw for insertion into the threaded hole and further into the ulna, wherein the second screw includes a portion of the shaft near a head to be threaded.

In yet another embodiment of the present invention, the system above for performing an ulnar shortening osteotomy further includes a cutting guide for guiding a cutting tool in cutting a portion of the ulna, wherein the cutting guide is coupled with the plate.

The foregoing and other features and advantages of the present invention will be apparent from the following more particular description of the preferred embodiments of the invention, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings. Additionally, the left-most digit of a reference number identifies the drawing in which the reference number first appears.

FIG. 1 is an illustration of a perspective view of an ulna plate positioned on the dorsum of an ulna, according to one embodiment of the present invention.

FIG. 2 is an illustration of a more detailed view of the ulna plate of FIG. 1.

FIG. 3 is an illustration of a cross sectional view of the ulna plate and the ulna of FIG. 1.

FIG. 4 is an illustration of a transverse sectional view of the ulna plate and ulna of FIG. 1.

FIG. 5A is an illustration of a detail of the ulna plate of FIG. 1.

FIG. 5B is an illustration of a cross sectional view of elliptical hole 40 of FIG. 5A.

FIG. 5C is an illustration of a top cross sectional view of elliptical hole 40 of FIG. 5A, including the screw 502.

FIG. 6 is an illustration of a perspective view of a cutting guide for use with the ulna plate of FIG. 1, in accordance with one embodiment of the present invention.

FIG. 7 is an illustration of another perspective view of the cutting guide of FIG. 6.

FIG. 8 is an illustration of yet another perspective view of the cutting guide of FIG. 6.

FIG. 9 is an illustration of a perspective view of an alternative version of the cutting guide of FIG. 6.

FIG. 10 is an illustration of a perspective view of the ulna plate and the ulna of FIG. 1, including screws before insertion.

FIG. 11 is an illustration of a perspective view of the ulna plate and the ulna of FIG. 1, including screws after insertion.

FIG. 12 is an illustration of a perspective view of the ulna plate and the ulna of FIG. 1, including the cutting guide of FIG. 6.

FIG. 13 is an illustration of a perspective view of the ulna plate and the ulna of FIG. 1, after a single bone cut.

FIG. 14 is an illustration of a perspective view of the ulna plate and the ulna of FIG. 1, during extraction of a section of the ulna.

FIG. 15 is an illustration of a perspective view of the ulna plate and the ulna of FIG. 1, after extraction of the section of the ulna.

FIG. 16 is an illustration of a perspective view of a bone rasp, in accordance with one embodiment of the present invention.

FIG. 17 is an illustration of a perspective view of a compression guide for use with the ulna plate of FIG. 1, in accordance with one embodiment of the present invention.

FIG. 18 is an illustration of a perspective view of the ulna plate and the ulna of FIG. 1, before coupling with the compression guide.

FIG. 19 is an illustration of a perspective view of the ulna plate and the ulna of FIG. 1, during coupling with the compression guide.

FIG. 20 is an illustration of a perspective view of a compression clamp for use with the ulna plate of FIG. 1, in accordance with one embodiment of the present invention.

FIG. 21 is an illustration of a perspective view of the ulna plate and the ulna of FIG. 1, while coupled with the compression guide and compression clamp.

FIG. 22 is an illustration of a perspective view of the ulna plate and the ulna of FIG. 1, after compression with the compression guide and compression clamp.

FIG. 23 is an illustration of a perspective view of the ulna plate and the ulna of FIG. 1, after compression.

FIG. 24 is an illustration of a perspective view of the ulna plate and the ulna of FIG. 1, after insertion of a lag screw.

### DETAILED DESCRIPTION

In one embodiment of the present invention, a system for performing an ulnar shortening osteotomy is disclosed. The system includes an elongated metallic plate having tapered ends, wherein the plate is concave in a direction perpendicular to a main axis of the plate. The ulnar osteotomy plate includes a plurality of holes along the main axis of the plate, wherein the plurality of holes includes a substantially elliptical hole with a beveled interior edge located on a first side of the plate and a threaded hole located on a second side of the plate. The system also includes a first screw for insertion into one end of the elliptical hole and further into an ulna underneath the plate, wherein the first screw includes a beveled edge underneath a head such that when the beveled edge of the first screw contacts the beveled interior edge of the elliptical hole, there results a force along the main axis towards the first side. The system also includes a second screw for insertion into the threaded hole and further into the ulna, wherein the second screw includes a portion of the shaft near a head to be threaded. In another embodiment of the present invention, the system above for performing an ulnar shortening osteotomy further includes a cutting guide for guiding a cutting tool in cutting a portion of the ulna, wherein the cutting guide is coupled with the plate.

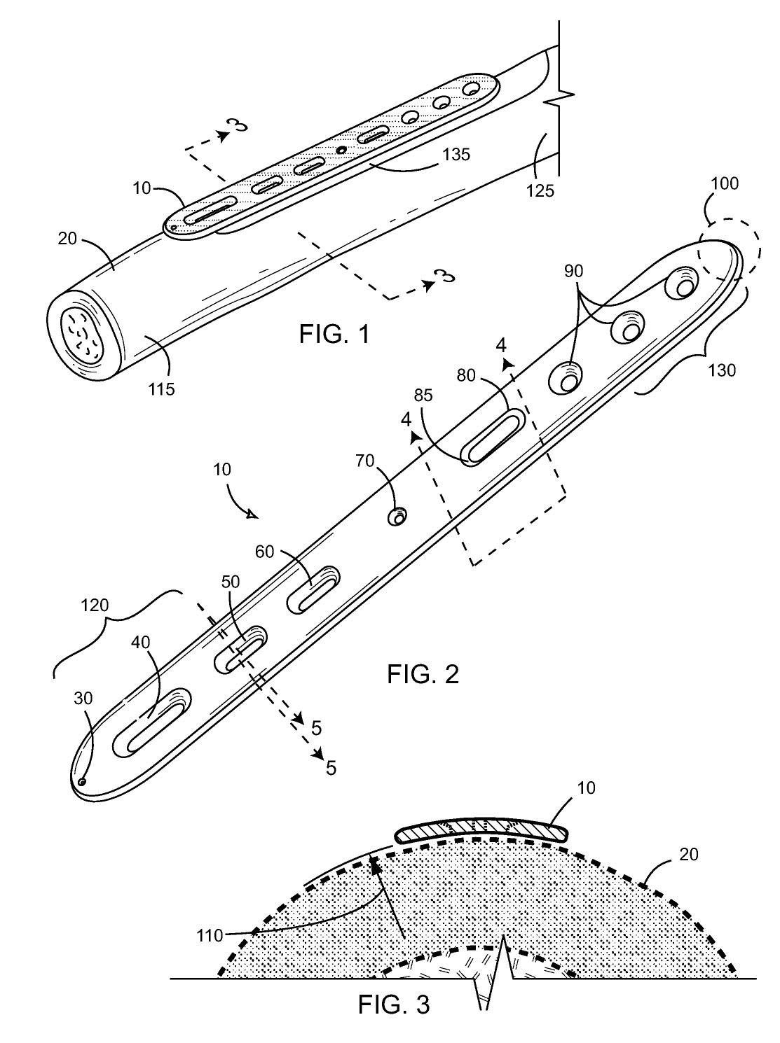

FIG. 1 is an illustration of a perspective view of an ulna plate 10 positioned on the dorsum 135 of an ulna 20, according to one embodiment of the present invention. The ulna 20 comprises a distal end 125 and a proximal end 115. The ulna plate 10 comprises an elongated metallic plate having tapered ends, wherein the ulna plate 10 is slightly concave in a direction perpendicular to a main axis of the ulna plate 10.

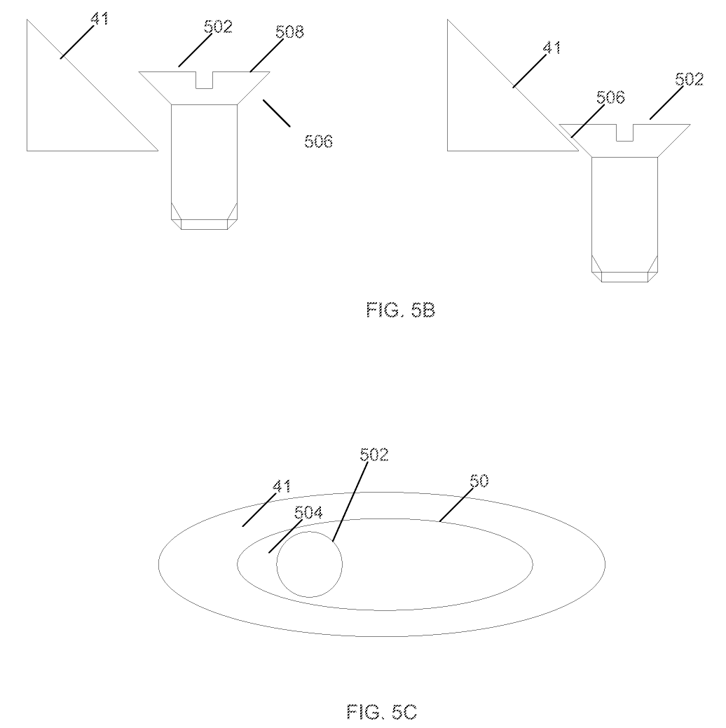

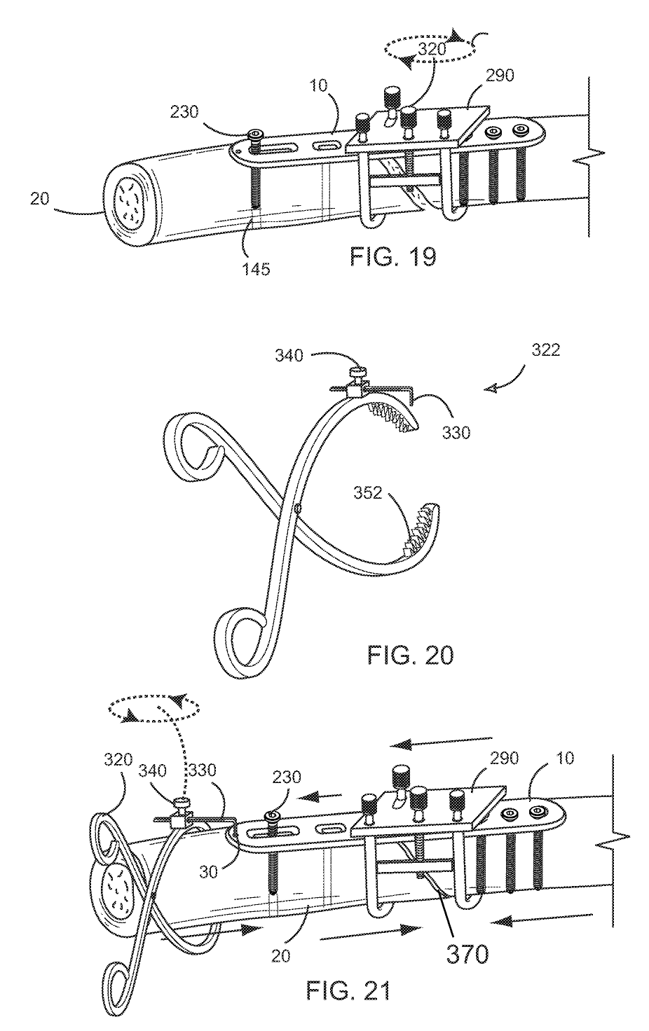

FIG. 2 is an illustration of a more detailed view of the ulna plate 10 of FIG. 1. The ulna plate 10 comprises an elongated metallic plate having tapered ends, such as end 100. The ulna plate 10 includes holes 30, 40, 50, 60 located on the main axis of the proximal side 120 of the ulna plate 10. Hole 40 is an elongated hole that allows a screw 230 to slide across its orifice, as described below in greater detail with reference to FIGS. 19-21. Holes 50 and 60 comprise substantially elliptical holes with a beveled interior edge, as described in greater detail below with reference to FIGS. 5A-5C. Hole 30 is uniform-width service hole used by the compression clamp 320, as described in greater detail below with reference to FIG. 21.

The ulna plate 10 also includes three holes 90 located on the main axis of the distal side 130 of the ulna plate 10. The most distal hole of the three holes 90 is a locking hole that includes a threaded interior so as to allow a threaded screw to be secured to the locking hole, wherein the screw is also screwed into the ulna bone 20.

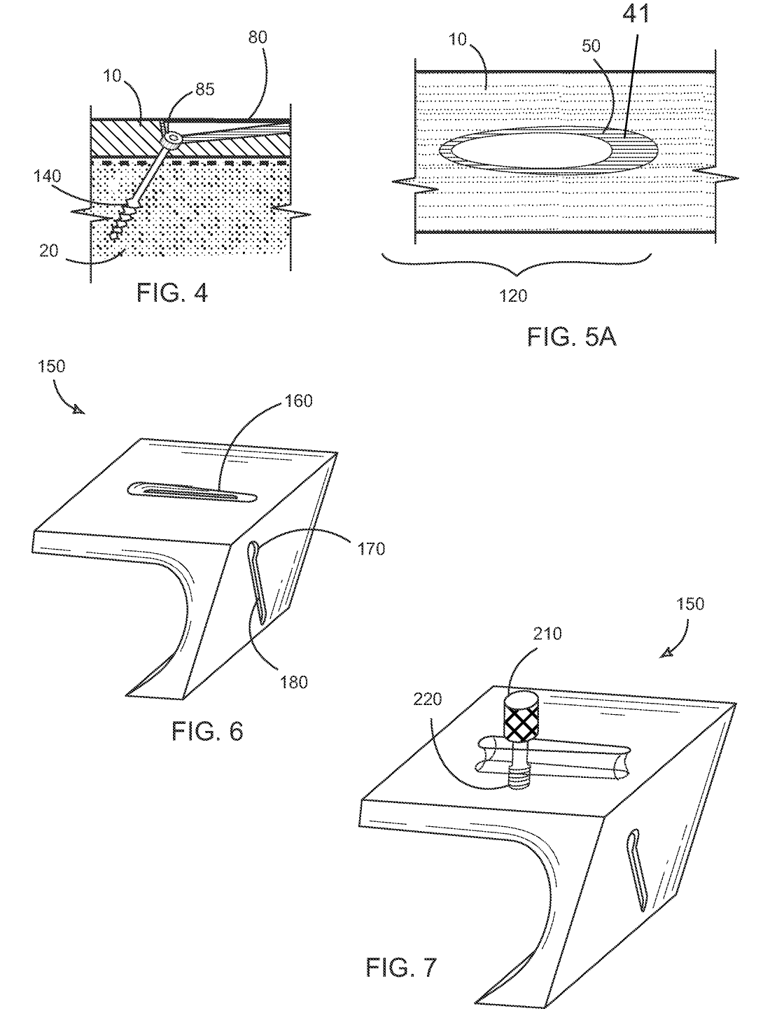

Hole 70, located on the main axis at the midpoint of the ulna plate 10, is a smaller sized threaded hole used by the compression guide 290 and cutting guides 150, 200, described in greater detail below. Elongated hole 80, located on the main axis of the distal side 130 of the ulna plate 10, is a lengthened or extended hole with a countersink 85 used with a lag screw 140, described in greater detail below with reference to FIG. 4.

FIG. 3 is an illustration of a cross sectional view of the ulna plate 10 and the ulna 20 of FIG. 1. Note the ulna plate 10 has a slightly concave shape 110 in a direction perpendicular to a main axis of the ulna plate 10, so as to fit the curved shape of the ulna 20. Also note that the dimensions and curvatures of FIG. 3 are not to scale and have been exaggerated so as to illustrate a concept.

FIG. 4 is an illustration of a transverse sectional view of the ulna plate 10 and ulna 20 of FIG. 1. FIG. 4 shows elongated or lag screw hole 80, located on the main axis of the distal side 130 of the ulna plate 10. Hole 80 is a lengthened or extended hole with a countersink 85 used with a lag screw 140 inserted at an angle through hole 80 and into bone 20, described in greater detail below. A lag screw is a screw that produces compression on fixation because of partial threading (at the distal part of the screw). A lag screw is inserted across a fracture so as to provide compression across the fracture.

FIG. 5A is an illustration of a detail of the ulna plate 10 of FIG. 1. FIG. 5A shows a substantially elliptical hole 50 located on the main axis of the proximal side 120 of the ulna plate 10, wherein hole 50 includes a beveled or angled interior edge 41.

FIG. 5B is an illustration of a cross sectional view of elliptical hole 50 of FIG. 5A. FIG. 5B shows a screw 502 for insertion into a first end 504 of the elliptical hole 50 and further into the ulna 20 underneath the ulna plate 10. The screw 502 includes a beveled edge 506 underneath the head 508 such that as the screw 502 is driven into the hole 50, the beveled edge 506 of the screw 502 contacts the beveled interior edge 41 of the elliptical hole 50, thereby resulting in the creation of a force separating the screw 502 from the interior edge 41. Because the screw 502 is affixed or secured to a certain extent to the ulna 20, as the screw 502 is driven into the hole 50, the beveled edge 506 of the screw 502 contacts the beveled interior edge 41 of the elliptical hole 50, thereby resulting in a force against the interior edge 41 causing minute movement of the ulna plate 10 towards the proximal side 120, thereby further causing compression of the two cut bone ends, as explained in greater detail below.

FIG. 5C is an illustration of a top cross sectional view of elliptical hole 50 of FIG. 5A, including the screw 502. FIG. 5C shows a top cross section of the shaft of the screw 502 inserted into a first end 504 of the elliptical hole 50 and further into the ulna 20 underneath the ulna plate 10. Note the top cross section of the shaft of the screw 502 fits within the area of the hole 50 provided at the first end 504 of the elliptical hole 49. The top cross section of the beveled edge 506 of the screw 502, however, is larger than the area of the hole 50 provided at the first end 504 of the elliptical hole 50. Thus, as the screw 502 is driven into the hole 50, the beveled edge 506 of the screw 502 contacts the beveled interior edge 41 of the elliptical hole 50, thereby resulting in the creation of a force pushing the screw 502 from the interior edge 41 towards the center of the hole 50 where there is more room to accommodate the top cross section of the beveled edge 506 of the screw 502. Because the screw 502 is affixed or secured to a certain extent to the ulna 20, as the screw 502 is driven into the hole 50, the beveled edge 506 of the screw 502 contacts the beveled interior edge 41 of the elliptical hole 50, thereby resulting in a force against the interior edge 41 causing minute movement of the ulna plate 10 towards the proximal side 120, thereby further causing compression of the two cut bone ends.

FIG. 6 is an illustration of a perspective view of a cutting guide 150 for use with the ulna plate 10 of FIG. 1, in accordance with one embodiment of the present invention. The cutting guide 150 includes a slotted hole 160 for a thumb screw 210. The thumb screw 210 includes a threaded shaft 220 for insertion into a threaded hole. The cutting guide 150 further includes a slot 180 for a saw and a slotted hole 170 for a drill. The slot 180 is an elongated parallel slit that ends in a rounded hole 170. FIG. 7 is an illustration of another perspective view of the cutting guide 150 of FIG. 6.

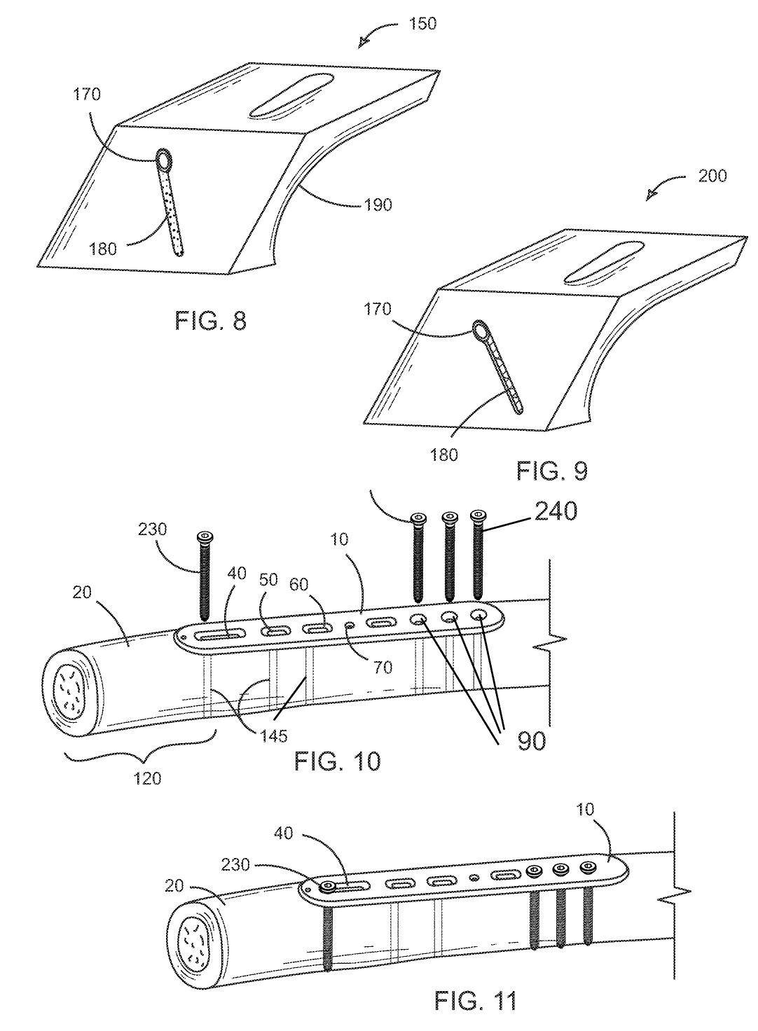

FIG. 8 is an illustration of yet another perspective view of the cutting guide 150 of FIG. 6. FIG. 8 also shows a slot 180 for a saw and a slotted hole 170 for a drill. FIG. 8 also shows the bone fitting concave shape 190 of the cutting guide 150. FIG. 9 is an illustration of a perspective view of an alternative version 200 of the cutting guide of FIG. 6. FIG. 9 shows a slot 180 and a slotted hole 170 positioned in a different position from the slot and hole of FIG. 8.

FIG. 10 is an illustration of a perspective view of the ulna plate 10 and the ulna 20 of FIG. 1, including screws 230, 240 before insertion. FIG. 10 shows the initiation of the method of performing an ulnar shortening osteotomy. Prior to the placement of the ulna plate 10 on the ulna 20, the skin, tissue and other elements of a patient's forearm are dissected and prepared for implantation of the ulna plate 10. Then, the ulna plate 10 is held to the dorsal (or volar) aspect of the ulna 20 with plate holding clamps. Once it has been determined that the ulna plate 10 is in the proper position on the ulna 20, screw holes are drilled into the ulna 20 for the three screws that enter holes 90 with a 2.3 mm-2.5 mm diameter drill bit using a tissue protection device. Also, holes 145 are drilled for holes 40, 50, 60 of ulna plate 10.

Then, the ulna plate 10 is secured to the ulna 20 by placing the three screws (approximately 3.2-3.5 mm) in the holes 90 of the ulna plate 10 and further into the holes that were drilled into the ulna 20. The distal most screw 240 may be a locking screw that locks into the ulna plate 10 via the distal most hole 90 of the ulna plate 10, the interior surface of which is threaded. In this case, the distal most screw 240 includes a portion of the shaft near the head that is threaded so as to thread into the threaded distal most hole 90.

Next, the most proximal screw 230 is placed in the most proximal location in hole 40. FIG. 11 is an illustration of a perspective view of the ulna plate 10 and the ulna 20 of FIG. 1, including screws 230, 240 after insertion.

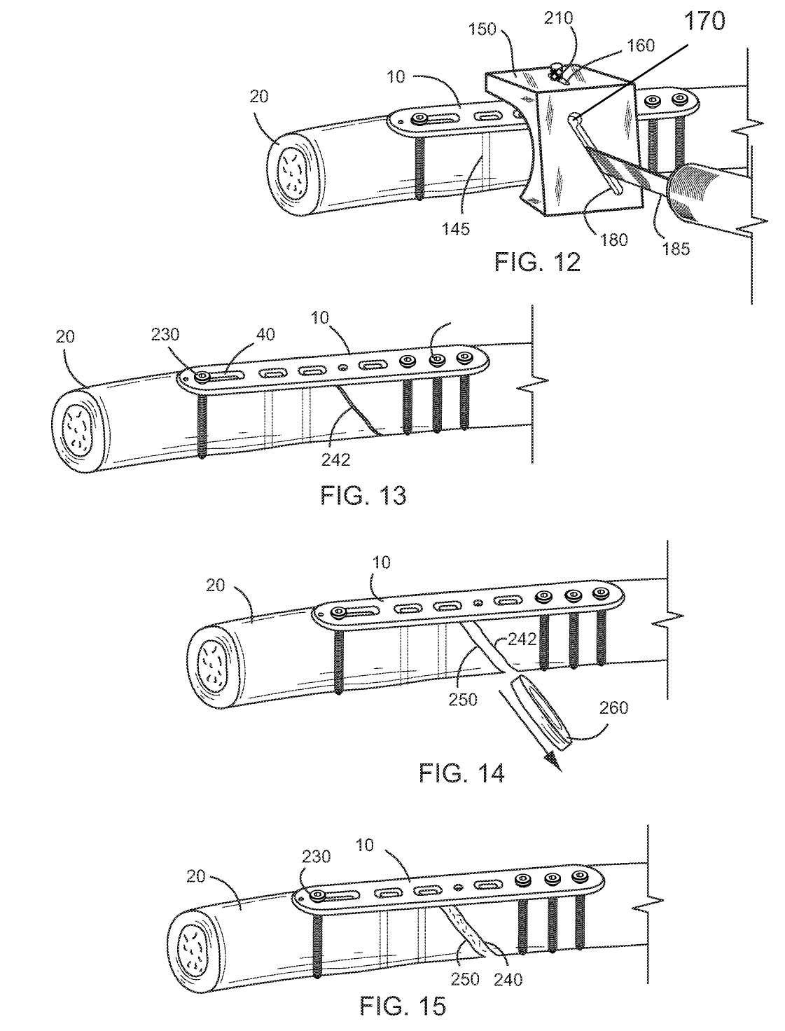

FIG. 12 is an illustration of a perspective view of the ulna plate 10 and the ulna 20 of FIG. 1, including the cutting guide 150 of FIG. 6. Next, the cutting guide 150 is attached to the ulna plate 10 by screwing thumb screw 210 into the hole 70 in the ulna plate 10. Hole 70 is a threaded hole that allows a threaded screw to be screwed into it, so as to secure a threaded screw to hole 70. A drill may be used within slotted hole 170 to cut away a portion of the ulna 20 beneath the concave portion of the ulna plate 10. The rest of the first cut 242 is created by inserting an oscillating or reciprocating saw into slot 180. Subsequently, the cutting guide 150 is removed. FIG. 13 is an illustration of a perspective view of the ulna plate 10 and the ulna 20 of FIG. 1, after a single bone cut 242.

Next, a second cutting guide, such as cutting guide 200, which has a parallel offset slot 180 so as to create a parallel cut 2-5 mm from the first cut, is attached to the ulna plate 10 by screwing thumb screw 210 into the hole 70 in the ulna plate 10. A drill may be used within slotted hole 170 to cut away a portion of the ulna 20 beneath the concave portion of the ulna plate 10. The rest of the second cut 250 is created by inserting an oscillating or reciprocating saw into slot 180, thereby creating a slice of bone 260. FIG. 14 is an illustration of a perspective view of the ulna plate 10 and the ulna 20 of FIG. 1, during extraction of the slice of bone 260. FIG. 15 is an illustration of a perspective view of the ulna plate 10 and the ulna 20 of FIG. 1, after extraction of the section of the ulna.

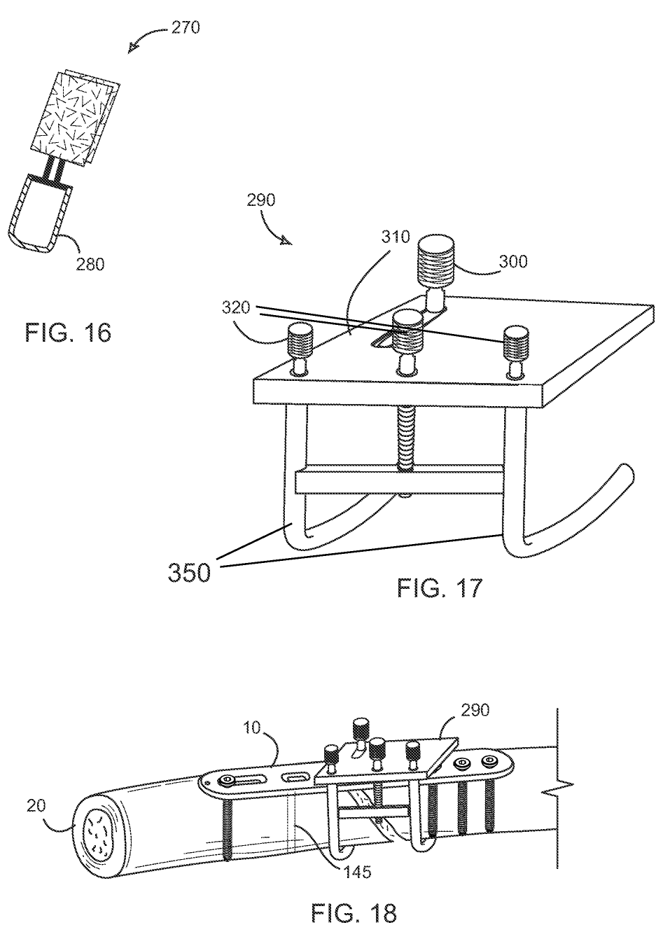

FIG. 16 is an illustration of a perspective view of a bone rasp 270, in accordance with one embodiment of the present invention. The bone rasp 270 includes a handle 280 with a rough working end, such as with a sandpaper texture, so as to smooth the edges of the cuts 242, 250.

FIG. 17 is an illustration of a perspective view of a compression guide 290 for use with the ulna plate 10 of FIG. 1, in accordance with one embodiment of the present invention. The compression guide 290 includes a planar element 310 for resting on top of the ulna plate 10 and the ulna 20. The planar element 310 includes two arms 350 that extend from the bottom and wrap around underneath the planar element 310, so as to grab or grip the ulna 20. The compression guide 290 also includes a set of screws 320 which adjust the distance or amount of play between the planar element 310 and the arms 350. The screws 320 adjust the tightness of the grip of the compression guide 290 upon the ulna plate 10 and the ulna 20. Also included is a threaded screw 300 that extends through the planar element 310 for coupling with threaded hole 70 of the ulna plate 10.

FIG. 18 is an illustration of a perspective view of the ulna plate 10 and the ulna 20 of FIG. 1, before coupling with the compression guide 290. FIG. 18 shows that compression guide 290 is placed on the ulna plate 10 and the ulna 20 so as to grip both items and compression them together. FIG. 19 is an illustration of a perspective view of the ulna plate 10 and the ulna 20 of FIG. 1, during coupling with the compression guide 290. FIG. 19 shows that the screws 320 are turned so as to adjust the tightness of the grip of the compression guide 290 upon the ulna plate 10 and the ulna 20. Also, the threaded screw 300 is threaded into threaded hole 70 of the ulna plate 10 so as to secure the compression guide 290 to the ulna plate 10. Subsequently, screw 230 is loosed so as to allow travel of the ulna plate 10.

FIG. 20 is an illustration of a perspective view of a compression clamp 322 for use with the ulna plate 10 of FIG. 1, in accordance with one embodiment of the present invention. The compression clamp 322 is a scissor or clamp-like device that includes handles 342, clamp ends 352 with teeth, a hook 330 with gear teeth and a hook worm gear 340 for adjusting the distance of the hook 330 from the compression clamp 322.

FIG. 21 is an illustration of a perspective view of the ulna plate 10 and the ulna 20 of FIG. 1, while coupled with the compression guide 290 and compression clamp 322. FIG. 21 shows that the clamp ends 352 of the compression clamp 322 have gripped the ulna 20 and the hook 330 has been attached to the hole 30 of the ulna plate 10. Hole 30 is an unthreaded uniform-width hole with dimensions that accommodate the hook 330. The hook worm gear 340 is then turned so as to adjust the distance of the hook 330 from the compression clamp 322. This causes the left portion of the ulna 20 to move towards to the right portion and causes the right portion of the ulna 20 to move towards to the left portion. Thus, activation of the compression clamp 322 causes compression of the two cut ends of the ulna 20 at the juncture 370. This causes screw 230 to travel within the orifice of hole 40.

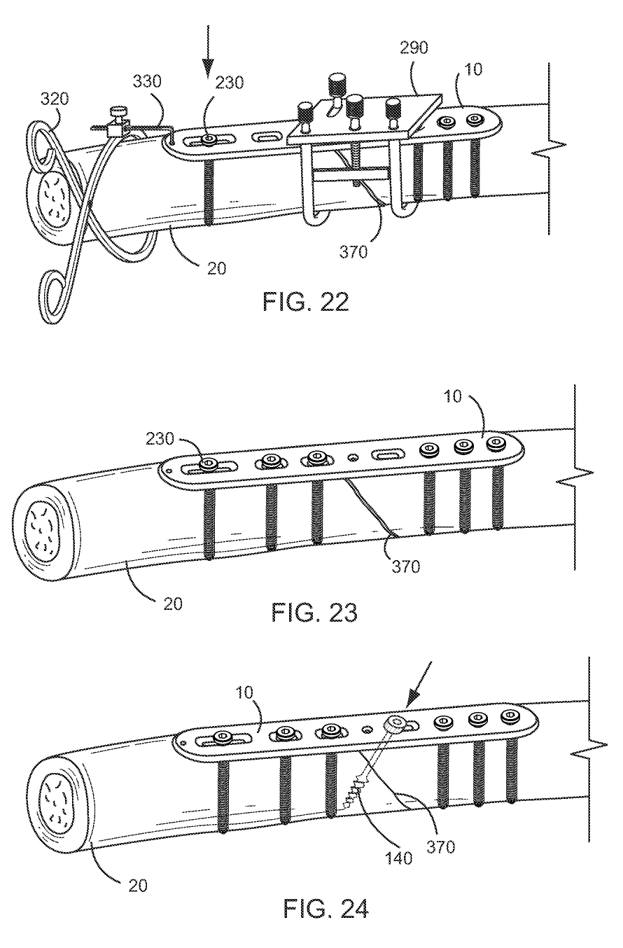

FIG. 22 is an illustration of a perspective view of the ulna plate 10 and the ulna 20 of FIG. 1, after compression at juncture 370 with the compression guide 290 and compression clamp 320. When enough compression has been attained, the screw 230 is tightened or driven into the ulna 20 so as to disallow travel of the ulna plate 10. FIG. 23 is an illustration of a perspective view of the ulna plate 10 and the ulna 20 of FIG. 1, after compression. FIG. 23 shows that the compression guide 290 and compression clamp 322 have been removed. Also, screws have been inserted into holes 50 and 60. FIG. 24 is an illustration of a perspective view of the ulna plate 10 and the ulna 20 of FIG. 1, after insertion of a lag screw 140, which is the final step of the operation. Before the lag screw 140 is inserted, a hole may be drilled into the appropriate location of the screw 140, such as with a 2.3-2.5 mm drill bit.

The present invention, as described above, overcomes the problems with the prior art and provides a more efficient way to perform an ulnar shortening osteotomy and reduce negative side effects of the surgery. The system of the present invention allows the surgeon to maintain the ulna and the cutting tool in a fixed position during cutting such that the two remaining surfaces of the cut bone are uniform and planar. The system of the present invention further allows the two cut bone ends to be aligned and kept from rotating during merging of the two ends, so as to ensure a proper attachment position.

The system of the present invention further reduces the number of devices or parts necessary to perform the ulnar shortening osteotomy, thereby simplifying the surgery for the surgeon and decreasing the risk of misuse and/or user error. The system of the present invention further provides for the secure attachment of the cutting guide to the ulna plate, so as to maintain an extremely secure connection and reduce the risk of the cutting guide falling out when vibrations are experienced.

The system of the present invention further provides increased compression of the two ends of bone in order to ensure proper healing of the two cut bone ends and foster the formation of bone mass at the junction point. The elliptical holes and the beveled edges of the holes 50, 60 of the ulna plate 10, along with the beveled edge of the screw heads, provide added compression to the two cut bone ends.

The system of the present invention further eliminates the drilling of holes in the ulna which are not filled at the end of the ulnar shortening osteotomy, thereby reducing weakness in the ulna bone, and infections or other complications. The system of the present invention further eliminates the need to add and remove screws multiple times from the ulna bone with the possibility of stripping the bone and losing purchase of the screws. The system of the present invention further facilitates reaching the section of bone underneath the curved ulna plate when cutting the ulna.

Lastly, the system of the present invention allows for the placement of the ulna plate on the dorsum of the ulna—a location of the human arm subject to little contact and impact during everyday use. This allows the ulna plate to remain under soft tissue coverage away from areas of normal impact on the ulna midaxial line of the forearm where previous ulna plates were designed to fit. By moving the ulna plate away from frequent impact areas and into a region of the forearm with good soft tissue coverage there is less chance that the patient will feel the ulna plate. The ulna plate is carefully rounded and tapered at the proximal and distal ends so there will be little interference with overlying tendons. This allows the ulna plate to remain in place indefinitely in many patients. This therefore eliminates the need for a second surgery to remove painful hardware, obviates the need for a second course of immobilization, and eliminates or reduces the cases of late fracture through the osteotomy site or an old screw hole once the ulna plate has been removed.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

We claim:

1. An ulnar osteotomy plate, comprising:
an elongated metallic plate having tapered ends, wherein the plate is concave in a direction perpendicular to a main axis of the plate; and
a plurality of holes along the main axis of the plate, comprising:
a substantially elliptical hole with a beveled interior edge located on a first side of the plate; and
a threaded hole located on a second side of the plate.

2. The ulnar osteotomy plate of claim 1, wherein the plurality of holes comprises three holes on the first side and four holes on the second side.

3. The ulnar osteotomy plate of claim 2, wherein each of the three holes on the first side comprise a substantially elliptical hole with a beveled interior edge.

4. The ulnar osteotomy plate of claim 3, wherein the three holes on the first side vary in size.

5. The ulnar osteotomy plate of claim 3, wherein one of the four holes on the second side comprise a threaded hole.

6. A system for performing an ulnar osteotomy, comprising:
an elongated metallic plate having tapered ends, wherein the plate is concave in a direction perpendicular to a main axis of the plate;
a plurality of holes along the main axis of the plate, comprising:
a substantially elliptical hole with a beveled interior edge located on a first side of the plate; and
a threaded hole located on a second side of the plate;
a first screw for insertion into one end of the elliptical hole and further into an ulna underneath the plate, wherein the first screw includes a beveled edge underneath a head such that when the beveled edge of the first screw contacts the beveled interior edge of the elliptical hole, there results a force along the main axis towards the first side; and
a second screw for insertion into the threaded hole and further into the ulna, wherein the second screw includes a portion of the shaft near a head to be threaded.

7. The system of claim 6, wherein the plurality of holes comprises three holes on the first side and four holes on the second side.

8. The system of claim 7, wherein each of the three holes on the first side comprise a substantially elliptical hole with a beveled interior edge.

9. The system of claim 8, wherein the three holes on the first side vary in size.

10. The system of claim 8, wherein one of the four holes on the second side comprise a threaded hole.

11. The system of claim 7, further comprising:
a third and fourth screw, each having a beveled edge underneath a head, each for insertion into one of the three holes on the first side; and
a fifth, sixth, seventh and eighth screw, each for insertion into one of the four holes on the second side.

12. The system of claim 6, wherein the first screw further comprises a tapered shaft that is threaded for insertion into an ulna.

13. The system of claim 12, wherein the second screw further comprises a tapered shaft that is threaded for insertion into an ulna and a portion of the shaft near the head is threaded for contact with the threaded hole.

14. A system for performing an ulnar osteotomy, comprising:
an elongated metallic plate having tapered ends, wherein the plate is concave in a direction perpendicular to a main axis of the plate;
a plurality of holes along the main axis of the plate, comprising:
a substantially elliptical hole with a beveled interior edge located on a first side of the plate; and
a threaded hole located on a second side of the plate;
a first screw for insertion into one end of the elliptical hole and further into an ulna underneath the plate, wherein the first screw includes a beveled edge underneath a head such that when the beveled edge of the first screw contacts the beveled interior edge of the elliptical hole, there results a force along the main axis towards the first side;
a second screw for insertion into the threaded hole and further into the ulna, wherein the second screw includes a portion of the shaft near a head to be threaded; and
a cutting guide for guiding a cutting tool in cutting a portion of the ulna, wherein the cutting guide is coupled with the plate.

15. The system of claim 14, wherein the plurality of holes comprises three holes on the first side, wherein each of the three holes on the first side comprise a substantially elliptical hole with a beveled interior edge, and four holes on the second side, wherein one of the four holes on the second side comprise a threaded hole

16. The system of claim 14, further comprising:
a second threaded hole in the plate; and
a second threaded screw in the cutting guide, wherein the second threaded screw attaches to the second threaded hole so as to secure the cutting guide to the plate.

17. The system of claim 16, further comprising:
a compression guide for guiding the merging of two pieces of the ulna that are separated by a cut, the compression guide comprising:
a planar element for placement on top of the plate;
at least one arm that extends from the planar element underneath the ulna;
at least one screw that adjusts the distance between the planar element and the at least one arm; and
at least one screw that couples the compression guide to the plate.

18. The system of claim 17, further comprising:
a compression clamp for merging the two pieces of the ulna that are separated by the cut, the compression clamp comprising:
a handheld clamp that clamps one piece of the ulna;
a hook extending from the clamp for attachment to the plate; and
a gear that pulls the hook towards the clamp when activated.
